(19) United States
(12) Patent Application Publication
(10) Pub. No.: US 2011/0129567 A1
(43) Pub. Date: Jun. 2, 2011

(54) NOVEL BREADMAKING YEAST STRAINS

(75) Inventors: Didier Jacques Colavizza, Roubaix (FR); Anne-Dominique Madeleine Quipourt-Isnard, Marcq-En-Baroeul (FR)

(73) Assignee: LESAFFRE ET COMPAGNIE, Paris (FR)

(21) Appl. No.: 12/674,854

(22) PCT Filed: Aug. 14, 2008
(86) PCT No.: PCT/FR2008/001193

§ 371 Date: Feb. 23, 2010

(30) Foreign Application Priority Data
Aug. 23, 2007 (FR) 0705977

(51) Int. Cl. A21D 13/00; C12N 1/18; C12Q 1/04; A21D 10/00

(52) U.S. Cl. 426/18; 435/255.2; 435/34; 426/62; 426/549

(57) ABSTRACT

The disclosure relates to novel baker's yeast strains, fresh and dry baker's yeasts obtained from said strains and the use thereof in bread-making, said strains being effecting on sugar-free doughs and/or lightly sweetened doughs, optionally in the presence of mould inhibitors.

### CROSS-REFERENCE TO RELATED APPLICATIONS

This application is a National Phase Entry of International Application No. PCT/FR2008/001193, filed on Aug. 14, 2008, which claims priority to French Application 07 05 977, filed on Aug. 23, 2007, both of which are incorporated by reference herein.

### FIELD OF THE INVENTION

The present invention relates to novel performing strains of breadmaking yeast, also called baker's yeast, on non sweetened dough and on slightly sweetened dough.

### TECHNOLOGICAL BACKGROUND

In flours, the majority source of carbohydrates for the yeast is maltose from the cleaving of starch by amylases. However, baker's yeasts do not directly metabolize maltose. Maltose enters the yeast by active transport via a maltopermease and is then cleaved into two glucose molecules by an intracellular enzyme, maltase. The carbohydrates which may be immediately used by the yeast, notably simple sugars of the glucose or saccharose type, only represent 1% to 1.5% by mass of conventional flours.

A system of glucose repression of the expression of the genes coding for maltopermease and maltase is observed in yeast strains described as “slow strains”. In these slow strains, the presence of simple sugars of the glucose or saccharose type in the flour notably induces repression of the maltase activity. These yeasts in a first phase ferment the simple sugars of the glucose or saccharose type present in the flour. When the simple sugars are entirely consumed, the yeasts are not equipped with maltopermease and maltase for causing maltose to enter the cell and for degrading it into glucose. This is expressed by a reduction in the volume of CO₂ produced by the yeast. This reduction in the volume of CO₂ corresponds to the time for having the yeast set up the maltose degradation system, after induction of the expression of the genes of maltopermease and maltase by maltose. In so-called “rapid” yeast strains, there exists deregulation of the metabolism of maltose, also called “non-repression in the presence of glucose”, which is expressed by the existence of maltopermease and maltase activity in the presence of glucose.

In the breadmaking industry, there is a continuous need for having novel yeast strains with which baker's yeasts may be produced in different forms, with optimum yields and fermentative strengths, said yeasts being efficient on different types of dough. Such yeasts notably have advantages in industrial production where a limited number of yeasts may thus be used. However, it remains difficult to select strains having optimum properties at all levels and a parameter is often improved at the expense of another.

A reference baker's yeast strain used by the Applicant for several years is the strain deposited at the NCYC (National Collection of Yeast Cultures) under number NCYC995. This strain which is the subject-matter of U.S. Pat. No. 4,396,632 notably allows the production of baker's yeasts with excellent growth yields. However, although it is a rapid strain, the induction level of the maltase activity in the presence of maltose and the fermentative strengths of strain NCYC995 are not optimum relatively to those of other yeast strains which are however slow strains and/or which are not adapted to application on non sweetened or slightly sweetened dough. For example, the strain deposited on Mar. 22, 2000 at the CNCM under number I-2412 has a strong induction level of maltase activity in the presence of maltose, but this is a slow strain which is not adapted to making breads with non sweetened dough.

There is therefore a need for yeast strains which rapidly ferment the sugars by means of optimum non-repression of the maltase activity in the presence of glucose and/or of optimum induction of maltase activity in the presence of maltose, said strains having an improved fermentative strength.

### SUMMARY OF THE INVENTION

The object of the invention is therefore Saccharomyces cerevisiae strains deposited on Aug. 21, 2007 at the CNCM under numbers I-3796, I-3797 and I-3798, strains derived from these strains, strains obtained by crossing or transformation by mutation, or genetic transformation, of above strain(s). The object of the invention is also baker's yeasts which is obtainable by cultivation of an above strain and baker's dough containing these yeasts which according to an embodiment are not sweetened or are slightly sweetened. The invention is also directed to a method of preparing baker's dough comprising a step of fermentation by yeast according to the invention, and to the baking method, as well as to the breadmaking product thus obtained. The object of the invention is also a novel method of selecting baker's yeast strains.

### SHORT DESCRIPTION OF THE DRAWINGS

FIG. 1 represents the repression of the maltase activity in the presence of glucose and induction in the presence of maltose. In this figure, the white histograms represent the maltase activity in nanomoles of released p-nitrophenol per minute and per milligram of proteins under the repression condition and the grey histograms under the induction condition, for each tested strain: the reference strain NCYC995, the slow osmotolerant strain NCYC996, the slow strain CNCM I-2412 and the strains according to the invention deposited at the CNCM under the numbers I-3796, I-3797 and I-3798.

### DETAILED DESCRIPTION OF EMBODIMENTS

The present invention provides novel breadmaking yeast strains having better induction level of the maltase activity in the presence of maltose and/or better fermentative strength than the reference strain deposited under number NCYC995, while maintaining non-repression of the maltase activity in the presence of glucose characteristic of a rapid strain and/or of good yields during the production of yeasts (typically a yield greater than or equal to 90% of the yield obtained with the NCYC995 strain, preferably greater than or equal to 95%, still preferably greater than or equal to 98%). With the novel yeast strains according to the invention, yeasts having improved fermentative activity may notably be obtained. The advantages of the strains according to the invention appear in particular when the baker's yeasts obtained by cultivation of said strains are used as a fermentation agent in non sweetened or slightly sweetened dough, and possibly containing a mould inhibitor such as a weak organic acid and/or one of its salts. Three novel Saccharomyces cerevisiae strains obtained in this way were deposited on Aug. 21, 2007 in accordance with the Budapest Treaty at the CNCM (Collection Nationale de Cultures de Micro-organismes, Institut Pasteur, 25 rue du Docteur Roux, F-75724 Paris Cedex 15, France) under numbers I-3796, I-3797 and I-3798.

The yeast strains, object of the invention, were obtained by crossing strains having different profiles for inducing maltase activity in the presence of maltose and for repressing maltase activity in the presence of glucose, the goal being to select the strains having the best induction level in the presence of maltose, a non-repression in the presence of glucose characteristic of a rapid strain and the capability of producing yeasts with a good yield, said yeasts having a fermentative strength above that of yeasts from the Applicant's reference strain deposited under number NCYC995. The sporulation and crossing program was performed according to standard techniques, such as those taught in Chapter 7 “Sporulation and Hybridization of Yeast” by R. R. Fowell, from the reference textbook “The Yeasts”, Volume 1, edited by A. H. Rose and J. S. Harrison, 1969—Academic Press.

The yeast strains according to the invention are then selected using the following criteria:

1. Maltase activity under induction or repression conditions:
maltase activity in the presence of maltose greater than that of the strain NCYC995,
maltase activity in the presence of glucose characteristic of a rapid strain,

2. Yield and fermentative strength after cultivation on a molasses plate:
yield of the strain after cultivation on a molasses plate greater than or equal to 90% of the yield obtained with the NCYC995 strain,
fermentative strength of the strain after cultivation on a molasses plate greater than or equal to that of the NCYC995 strain,

3. Yield and fermentative strength after semi-continuous cultivation (fed-batch):
yield of the strain after semi-continuous cultivation greater than or equal to 90% of the yield obtained with the NCYC995 strain,
fermentative strength of the strain after semi-continuous cultivation greater than that of the NCYC995 strain,

4. Fermentative activity in breadmaking tests
fermentative activity of the yeasts from the breadmaking yeast strain greater than that of yeasts from the NCYC995 strain.

By preselecting the strains after cultivation on a molasses plate, it is possible to eliminate the strains having a too small yield relatively to that of the reference strain and/or a too low fermentative strength relatively to that of the reference strain. Indeed, cultivation on a molasses plate is much easier to implement than a semi-continuous cultivation. In order to approach industrial conditions for evaluating the abilities of the strains, a selection of preselected strains is carried out using the same criteria, i.e. yield and fermentative strength, but after semi-continuous cultivation.

The induction level of the maltase activity is defined here as the maltase activity in the presence of maltose and in the absence of glucose. In the following, the expression “maltase activity under induction conditions” involves the presence of maltose and the absence of glucose. The repression level of the maltase activity is defined here as the maltase activity in the presence of glucose and in the absence of maltose. In the following, the expression “maltase activity under repression conditions” involves the presence of glucose and the absence of maltose.

A level of non-repression of the maltase activity better than the reference strain deposited under number NCYC995 corresponds to a maltase activity in the presence of glucose greater than that of said reference strain. The maltase activity in the presence of maltose of the yeast strains according to the invention is preferably increased by at least 20%, notably by at least 30%, notably by at least 40%, notably by at least 50% and notably by at least 60% relatively to that of the NCYC995 strain. The maltase activity in the presence of glucose of the yeast strains according to the invention is greater than or equal to 50%, notably greater than or equal to 70%, notably greater than or equal to 90% of that of the NCYC995 strain. A maltase activity in the presence of glucose greater than or equal to 50% of the maltase activity of the reference strain is characteristic of a rapid strain.

In a preferred embodiment, the maltase activity in the presence of glucose of the yeast strains according to the invention is increased by at least 10%, preferably by at least 20% and still preferably by at least 30% relatively to that of the NCYC995 strain. The maltase activity is measured by standard techniques known to one skilled in the art. In particular, the maltase activity may be measured by assaying the release of a coloured product, p-nitrophenol, following the action of maltase on a chromogenic substrate, p-nitrophenyl-α-D-glucopyranoside, as described in Houghton-Larsen and Anders Brandt, Appl. Environ. Microbiol., 2006, pp. 7176-7182. Before assaying the maltase activity, the yeasts are incubated, notably for 4 hours, in a medium containing glucose for measuring the repression of maltase activity or containing maltose for measuring induction of maltase activity.

By “yield”, is designated here the growth yield of the yeasts. The yield is obtained by calculating the ratio of the produced yeast mass over the consumed sugar mass. The yield may be evaluated after cultivation on a molasses plate or after semi-continuous cultivation (fed-batch), as described in the reference textbook “Yeast Technology”, 2^{nd} edition, 1991, G. Reed and T. W. Nagodawithana, published by Van Nostrand Reinhold, ISBN 0-442-31892-8. Advantageously, with the strains according to the invention yeasts may be produced with a yield greater than or equal to the yield obtained with the NCYC995 strain.

The fermentative strength corresponds to the volume of CO₂ (in mL) produced by the yeast in fermentation in flour dough rolls. The fermentative strength is measured by standard techniques known to one skilled in the art, notably by means of a fermentometer as described by Burrows and Harrison in “Journal of the Institute of Brewing”, Vol. 65, 1959. In particular, the fermentative strength is measured according to tests described in EP 0511108 and U.S. Pat. No. 5,741,695 assigned to the applicant. The fermentative strength expresses the fermentative activity of glucose, it being understood that maltose and saccharose are always fermented as glucose or fructose.

The fermentative strength may be measured on yeasts obtained by cultivation of the strains on a molasses plate or by semi-continuous cultivation (fed-batch). The fermentative strength is preferably measured on fresh yeasts. The fermentative strength of the strains obtained after cultivation of the strains according to the invention on a molasses plate is greater or equal to that obtained with the NCYC995 strain. The fermentative strength of the yeasts obtained after semi-continuous cultivation of the strains according to the invention is preferably increased by at least 10%, notably by at least 14%, notably by at least 18%, notably by at least 22%, notably by at least 26% relatively to the fermentative strength of the NCYC995 strain.

Advantageously, the yeast strains according to the invention allow production of yeasts effective on non sweetened or slightly sweetened dough, supplemented or not with mould inhibitors, such as weak organic acids and/or their salts. Non sweetened dough is a dough in which no sugar has been added. In a non sweetened dough, the sugar present stems from the flour. The expression “slightly sweetened dough” designates dough having an added sugar content of less than or equal to 12%, notably less than or equal to 10%, notably less than or equal to 6%, notably less than or equal to 5%, notably less than or equal to 3% by mass relatively to the mass of the flour. The sugar is preferably added as saccharose.

Advantageously, the yeast strains according to the invention allow production of breadmaking yeasts having a fermentative activity greater than that of yeasts from the NCYC995 strain, either in the presence or not of at least one mould inhibitor. The fermentative activity may be evaluated during breadmaking by measuring the proof time. The proof time is a measurement currently used in the field of breadmaking. It is defined as the time required for the baker's dough to reach a certain height in the mould corresponding to the development of the desired dough before it is put into the oven. Owing to the yeasts produced by the different yeast strains according to the invention, the proof time, as measured in different bread recipes, is reduced.

Preferably, the yeasts obtained from yeast strains according to the invention have resistance to drying. With yeasts which are resistant to drying, it is possible to obtain dry yeasts. Drying is preferably careful rapid drying in the presence of an emulsifier. An emulsifier is notably 1.5% sorbitan monostearate. A yeast's resistance to drying is defined by a fermentative activity greater than or equal to 70% of the fermentative activity before drying, with constant dry matter, the fermentative activity being measured with the fermentometer of Burrows and Harrison in tests A1, A′1, A3, A′3 (in tests A3 and A′3, addition of 2 g of saccharose as compared with tests A1 and A′1) and under drying conditions described in documents EP 0511108 and U.S. Pat. No. 5,741,695.

The Saccharomyces cerevisiae strain deposited on Aug. 21, 2007 at the CNCM under number I-3796 notably has the following characteristics:
a maltase activity in the presence of maltose increased by at least 50% relatively to that of the NCYC995 strain,
a maltase activity in the presence of glucose greater than or equal to 50% of that of the NCYC995 strain,
during the production of yeasts, a yield greater than or equal to 90% of the yield obtained with the NCYC995 strain,
after cultivation on a molasses plate, a fermentative strength of the yeasts increased by at least 10% on a non sweetened dough relatively to that of yeasts from the NCYC995 strain, and
after semi-continuous cultivation, a fermentative strength of the yeasts increased by at least 14% on a non sweetened dough and by at least 26% on a slightly sweetened dough relatively to that of yeasts from the NCYC995 strain.

The Saccharomyces cerevisiae strain deposited on Aug. 21, 2007 at the CNCM under number I-3797 notably has the following characteristics:
a maltase activity in the presence of maltose increased by at least 60% relatively to that of the NCYC995 strain,
a maltase activity in the presence of glucose increased by at least 30% relatively to that of the NCYC995 strain,
during the production of yeasts, a yield greater than or equal to 90% of the yield obtained with the NCYC995 strain,
after cultivation on a molasses plate, a fermentative strength of the yeasts on a non sweetened dough greater than or equal than that of yeasts from the NCYC995 strain, and
after semi-continuous cultivation, a fermentative strength of the yeasts increased by at least 26% on a non sweetened dough and by at least 30% on a slightly sweetened dough relatively to that of yeasts from the NCYC995 strain.

The Saccharomyces cerevisiae strain deposited on Aug. 21, 2007 at the CNCM under number I-3798 notably has the following characteristics:
a maltase activity in the presence of maltose increased by at least 30% relatively to that of the NCYC995 strain,
a maltase activity in the presence of glucose greater than or equal to 50% of that of the NCYC995 strain,
during the production of yeasts, a yield greater than or equal to 90% of the yield obtained with the NCYC995 strain,
after cultivation on a molasses plate, a fermentative strength of the yeasts increased by at least 10% on a non sweetened dough relatively to that of yeasts from the NCYC995 strain, and
after semi-continuous cultivation, a fermentative strength of the yeasts increased by at least 14% on a non sweetened dough and by at least 26% on a slightly sweetened dough relatively to that of yeasts from the NCYC995 strain.
   The present invention relates to the three strains described above and to all the strains belonging to the same family, i.e. all the strains which share the same properties as the three strains described above, as well as all the strains which may be derived from this family of strains, and in particular from the three deposited strains.

An object of the invention is particularly a Saccharomyces cerevisiae strain derived from a strain as defined above, said derived strain being characterized by:
a maltase activity in the presence of maltose increased by at least 20% notably by at least 30% relatively to that of the NCYC995 strain, and/or
a maltase activity in the presence of glucose greater than or equal to 50% of that of the NCYC995 strain, and/or
during the production of yeasts, a yield greater than or equal to 90% of the yield obtained with the NCYC995 strain, and/or
after cultivation on a molasses plate, a fermentative strength of the yeasts on a non sweetened dough greater than or equal to that of yeasts from the NCYC995 strain, and/or
after semi-continuous cultivation, a fermentative strength of the yeasts on a non sweetened dough increased by at least 10%, notably by at least 14% relatively to that of yeasts from the NCYC995 strain, and/or
after semi-continuous cultivation, a fermentative strength of the yeasts on a slightly sweetened dough increased by at least 10%, notably by at least 14%, notably by at least 18%, notably by at least 22%, notably by at least 26% relatively to that of yeasts from the NCYC995 strain.

The invention is also particularly directed to a Saccharomyces cerevisiae strain derived from a strain as defined above, said derived strain being characterized by:
a maltase activity in the presence of maltose increased by at least 20%, notably by at least 30%, notably by at least 40%, notably by at least 50%, notably by at least 60% relatively to that of the NCYC995 strain, and/or
a maltase activity in the presence of glucose greater than or equal to 50%, notably greater than or equal to 70%, notably greater than or equal to 90% of that of the NCYC995 strain, and/or
during the production of yeasts, a yield greater than or equal to 90% of the yield obtained with the NCYC995 strain, preferably greater than or equal to 95%, still preferably greater than or equal to 98%, and/or
after cultivation on a molasses plate, a fermentative strength of the yeasts greater than or equal to that of the NCYC995 strain, and/or
after semi-continuous cultivation, a fermentative strength of the yeasts increased by at least 10%, notably by at least 14%, notably by at least 18%, notably by at least 22%, notably by at least 26% relatively to that of yeasts from the NCYC995 strain.

A strain derived from the I-3796 strain is preferably characterized by:
a maltase activity in the presence of maltose increased by a least 20%, notably by at least 30%, notably by at least 40%, notably by at least 50% relatively to that of the NCYC995 strain, and/or
a maltase activity in the presence of glucose greater than or equal to 50% of that of the NCYC995 strain, and/or
during the production of yeasts, a yield greater than or equal to 90% of the yield obtained with the NCYC995 strain, preferably greater than or equal to 95% and/or
after cultivation on a molasses plate, a fermentative strength of the yeasts on a non sweetened dough increased by at least 10% relatively to that of the NCYC995 strain, and/or
after semi-continuous cultivation, a fermentative strength of the yeasts on a non sweetened strain increased by at least 10%, notably by at least 14% relatively to that of the NCYC995 strain, and/or
after semi-continuous cultivation, a fermentative strength of the yeasts on a slightly sweetened dough increased by at least 10%, notably by at least 14%, notably by at least 18%, notably by at least 22%, notably by at least 26% relatively to that of the yeasts from the NCYC995 strain.

A strain derived from the I-3797 strain is preferably characterized by:

a maltase activity in the presence of maltose increased by a least 20%, notably by at least 30%, notably by at least 40%, notably by at least 50%, notably by at least 60% relatively to that of the NCYC995 strain, and/or
a maltase activity in the presence of glucose greater than or equal to 50%, notably greater than or equal to 70%, notably greater than or equal to 90% of that of the NCYC995 strain, and/or
during the production of yeasts, a yield greater than or equal to 90% of the yield obtained with the NCYC995 strain, and/or
after cultivation on a molasses plate, a fermentative strength of the yeasts on a non sweetened dough greater than or equal to that of the NCYC995 strain, and/or
after semi-continuous cultivation, a fermentative strength of the yeasts on a non sweetened dough increased by at least 10%, notably by at least 14%, notably by at least 18%, notably by at least 22%, notably by at least 26% relatively to that of the NCYC995 strain, and/or
after semi-continuous cultivation, a fermentative strength of the yeasts on a slightly sweetened dough increased by at least 10%, notably by at least 14%, notably by at least 18%, notably by at least 22%, notably by at least 26% relatively to that of the yeasts from the NCYC995 strain.

A strain derived from the I-3798 strain is preferably characterized by:
a maltase activity in the presence of maltose increased by a least 20%, notably by at least 30%, relatively to that of the NCYC995 strain, and/or
a maltase activity in the presence of glucose greater than or equal to 50% of that of the NCYC995 strain, and/or
during the production of yeasts, a yield greater than or equal to 90% of the yield obtained with the NCYC995 strain, and/or
after cultivation on a molasses plate, a fermentative strength of the yeasts on a non sweetened dough increased by at least 10% relatively to that of the NCYC995 strain, and/or
after semi-continuous cultivation, a fermentative strength of the yeasts on a non sweetened strain increased by at least 10%, notably by at least 14% relatively to that of the NCYC995 strain, and/or
after semi-continuous cultivation, a fermentative strength of the yeasts on a slightly sweetened dough increased by at least 10%, notably by at least 14%, notably by at least 18%, notably by at least 22%, notably by at least 26% relatively to that of the yeasts from the NCYC995 strain.

By the expression “derived strain”, is meant a strain derived by any transformation whatever it may be, such as for example by one or more crossings and/or by mutation and/or by genetic transformation. A strain derived by crossing is obtainable by crossing a strain according to the invention with the same strain or another strain according to the invention, or any other strain, for example the NCYC995 strain. A strain derived by mutation may be a strain which has been subjected to at least one spontaneous mutation in its genome or at least one mutation induced for example by mutagenesis. The mutation(s) of the derived strain is/are either silent or not. By the expression “mutagenesis”, is meant both standard mutagenesis obtained via radiations, for example by using UVs, or by mutagenic chemicals and insertional mutagenesis by transposition or integration of an exogenous DNA fragment. Mutagenesis via radiations comprises the use of UV, X or gamma radiations. Mutagenic chemicals agents are for example EMS (ethyl-methyl sulfonate), EES (ethyl-ethyl sulfonate), nitrosoguanidine, nitrous acid, aflatoxin B1, hydroxylamine, 5-bromo-uracil, 2-amino-purine, proflavine, orange acridine.

A strain derived by genetic transformation is a strain in which an exogenous DNA has been introduced. This exogenous DNA is preferably brought by a plasmid or directly integrated into the genome.

The invention is further directed to methods for transforming a strain as defined above, said method comprising a step of transforming said strain by mutagenesis or by genetic transformation. The present invention in particular relates to strains which are obtainable by the transformation method defined above. An object of the present invention is also a novel method of selecting breadmaking strains which includes an original combination of steps and with which yeast strains with the desired profile may be rapidly selected.

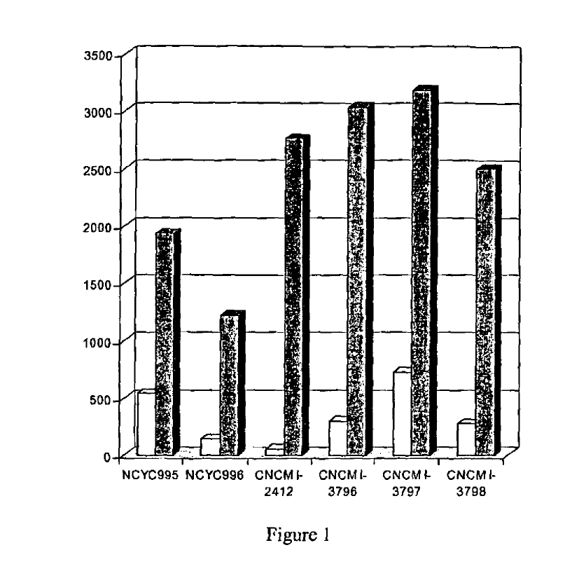

Surprisingly, the Applicant has shown that industrial yeast strains have very different maltase activity profiles, both under induction and repression conditions. For example, among the slow NCYC996 and CNCM I-2412 strains, the first one has a very low induction level in the presence of maltose (much lower than that of the rapid NCYC995 strain), while the induction level of the second one is very high (see FIG. 1). Still more surprisingly, the Applicant has shown that with a selection method including a step of selecting strains on the basis of maltase activity, it is possible to select strains which give yeasts with improved fermentative activity. This selection step on the basis of maltase activity consists of selecting strains, in which the maltase activity is weakly repressed in the presence of glucose and strongly induced in the presence of maltose. The selection method according to the invention further allows selection of breadmaking yeast strains which have a good yield.

An object of the present invention is thus a method of selecting improved breadmaking yeast strains, comprising the following steps:
a step of sporulation and hybridization of yeast strains and/or of mutagenesis of yeast strains, in order to obtain hybrid and/or mutated strains,
a first step of selecting hybrid and/or mutated strains which have:
   a maltase activity in the presence of maltose greater than that of the NCYC995 strain, and
   a maltase activity in the presence of glucose greater than or equal to 50% of the maltase activity of the NCYC995 strain,
      in order to obtain strains with improved maltase activity,
optionally, a step of pre-selecting strains, among the strains having improved maltase activity, which give yeasts, after cultivation on a molasses plate, which have:
   a yield greater than or equal to 90% of the yield obtained with the NCYC995 strain, and
   a fermentative strength greater than or equal to that of the

NCYC995 strain, and
a second step of selecting strains, among the strains having improved maltase activity and/or the strains obtained in the preceding step, which give yeasts, after semi-continuous cultivation, which have:
   a yield greater than or equal to 90% of the yield obtained with the NCYC995 strain, and
   a fermentative strength greater than or equal to that of the

NCYC995 strain,

in order to obtain improved breadmaking yeast strains.

Mutagenesis of yeast strains is notably carried out with the techniques described above. In an advantageous embodiment, the mutagenesis is a mutagenesis by the use of UV radiations or by the use of EMS.

Mutagenesis by use of UV radiations is for example carried out by preparing a culture of the strain to be mutated. The cell culture is then washed before being re-suspended in a buffer, for example a 0.9% NaCl solution. The cell suspension is then subjected to UV impacts, for example with radiation of 400 J/cm², according to the survival curve of the cells. The culture having been subjected to UV impacts is then spread onto a non-selective medium and each obtained colony corresponds to a mutated strain.

The selection method advantageously includes a pre-selection step. This pre-selection step is based on an original cultivation method of strains on plates of molasses. With this step a considerable amount of time may be saved in the selection process. Indeed, as cultivation on a molasses plate is easier to implement than semi-continuous cultivation, a larger number of hybrids may be tested over a short time.

An object of the invention is particularly a method as defined above, characterized in that:
the first selection step comprises the selection of hybrid and/or mutated strains which have:
   a maltase activity in the presence of maltose increased by at least 20%, notably by at least 30%, notably by at least 40%, notably by at least 50%, notably by at least 60% relatively to that of the NCYC995 strain, and/or
   a maltase activity in the presence of glucose greater than or equal to 70%, notably greater than or equal to 90% of that of the NCYC995 strain, and/or
the second selection step comprises the selection of strains giving yeasts, after semi-continuous cultivation, which have:
   a fermentative strength on a non sweetened and/or slightly sweetened dough increased by at least 10%, notably by at least 14%, notably by at least 18%, notably by at least 22%, notably by at least 26% relatively to that of the NCYC995 strain, and/or
   a yield greater than or equal to 95% of the yield obtained with the NCYC995 strain, preferably greater than or equal to 98%
      An object of the present invention is also a Saccharomyces cerevisiae strain which is obtainable by the selection methods as defined above.

An object of the present invention is also a Saccharomyces cerevisiae strain derived from a strain which is obtainable by the selection methods as defined above, said derived strain being characterized by:
a maltase activity in the presence of maltose increased by at least 20%, notably by at least 30% relatively to that of the NCYC995 strain, and/or
a maltase activity in the presence of glucose greater than or equal to 50% of that of the NCYC995 strain, and/or
during production of yeasts, a yield greater than or equal to 90% of the yield obtained with the NCYC995 strain, and/or
after cultivation on a molasses plate, a fermentative strength of the yeasts on a non sweetened dough greater than or equal to that of yeasts from the NCYC995 strain, and/or
after semi-continuous cultivation, a fermentative strength of the yeasts on a non sweetened dough increased by at least 10%, notably by at least 14% relatively to that of yeasts from the NCYC995 strain, and/or
after semi-continuous cultivation, a fermentative strength of the yeasts on a slightly sweetened dough increased by at least 10%, notably by at least 14%, notably by at least 18%, notably by at least 22%, notably by at least 26% relatively to that of the yeasts from the NCYC995 strain.

The invention is further directed to baker's yeast which is obtainable by cultivation of the strains as defined above, of derived strains and of transformed strains.

These breadmaking yeasts advantageously have at the industrial scale:
a yield greater than or equal to 90% of the yield obtained in the NCYC995 strain, preferably greater than or equal to 95%, still preferably greater than or equal to 98%, and/or
a resistance to drying greater than or equal to that of the NCYC995 strain, and/or
a breadmaking proof time shorter than the one obtained with breadmaking yeasts from the NCYC995 strain.
   The baker's yeasts according to the invention are produced from yeast strains as defined above, notably as described in the reference textbook “Yeast Technology”, 2nd edition, 1991, G. Reed and T. W. Nagodawithana, published by Van Nostrand Reinhold, ISBN 0-442-31892-8.

The making of baker's yeasts generally comprises at least the first two steps and the last step of the set of following steps:
multiplying a pure baker's yeast strain in several stages, first in semi-anaerobiosis, and then in aerobiosis,
separating the baker's yeast thus produced from its culture medium by centrifugation, thereby obtaining a liquid “yeast cream” containing about between 12 and 25% of dry matter, or even a larger amount of dry matter if the yeast cream is mixed with osmolyte products,
filtering the liquid yeast cream thus obtained, generally on a rotary filter in vacuo and obtaining a dehydrated fresh yeast containing from 26% to 35% of dry matter,
kneading said dehydrated fresh yeast in view of obtaining a very homogeneous mass,
extruding the yeast thus obtained and obtaining a pressed yeast as fresh yeast or crumbled fresh yeast blocks, marketed with about 30% dry matter, or, if the yeast is intended to be dried, as particles, generally granules,
optionally carefully drying the yeast particles obtained by extrusion, in a flow of hot air, for example by fluidization, in order to obtain dry yeast,
packaging.

In a particularly advantageous embodiment, the object of the present invention is baker's yeast as defined above, obtained by cultivating a yeast strain according to the invention with adaptation to the presence of a weak organic acid. Adaptation of the yeast according to the invention to the presence of a weak organic acid is notably carried out during the last multiplication stage by known methods, such as the method described in U.S. Pat. No. 4,318,991, with addition of 0.1 g to 10 g of short chain aliphatic carboxylic acids, such as aliphatic carboxylic acids with 2, 3 or 4 carbon atoms, and/or their salts, per litre of wort. This method of adaptation to the presence of a weak organic acid may optionally be combined with a method of the type described in U.S. Pat. No. 4,346,115, wherein, during the last cycle of multiplying the yeast, discontinuous pouring of molasses is applied, said discontinuous pouring preferably including short interruptions, for example: pouring molasses for 5-10 minutes followed by pouring interruptions of 5-10 minutes.

The baker's yeast may be yeast selected from yeast creams, pressed yeasts and dry yeasts. The yeast creams, pressed yeasts and dry yeasts are notably obtained by the method as defined above. Fresh baker's yeasts are characterized by high water content as compared with dry yeasts. Fresh baker's yeasts encompass yeast creams and pressed yeasts.

Yeast creams, also called “liquid yeasts”, are aqueous suspensions of yeast cells having a cream-type viscosity. By yeast cream, preferentially baker's yeast cream, is understood a liquid suspension, typically an aqueous suspension, of live, preferentially baker's, yeast cells, said suspension having a preferential dry matter content of at least 12% by mass and generally comprised between 12 and 50% by mass (extended definition of yeast cream). Preferably, liquid yeast cream meets the definition of yeast cream in the strict sense, i.e. it has a dry matter content between 12 and 25% by mass, and still preferably between 14 and 22% by mass. However, the present invention is also useful for (preferentially baker's) yeast creams with a higher dry matter content, i.e. of about 25% by mass, such as notably so-called high density baker's yeast creams containing one or more osmotic agents, such as for example food polyhydroxy compounds and food salts.

Among pressed yeasts, a distinction is made between yeasts pressed into a compact block, also called “yeast blocks”, characterized by 65%-74% water content and yeasts pressed into granules characterized by 63%-69% water content. Dry yeasts are characterized by low water content, notably less than 8% water. Dry yeasts comprise active dry yeasts and instantaneous dry yeasts. Active dry yeasts are yeasts which have to be re-hydrated in warm water before use. Instantaneous dry yeasts do not require the re-hydration step and may be directly added to the flour. An object of the present invention is more particularly baker's yeast as defined above, characterized in that the yeast is dry yeast, preferably instantaneous dry yeast.

The baker's yeasts according to the invention are adapted for use in dough at room temperature and/or cold dough. Room temperature dough here designates dough at a temperature comprised between 22° C. and 28° C., notably from 24° C. to 26° C. Cold dough here designates dough at a temperature greater than or equal to 15° C. and strictly lower than 22° C., notably dough at a temperature from 17° C. to 18° C.

The baker's yeasts according to the invention are adapted for a use in non sweetened or slightly sweetened dough. The bakery yeasts obtained with the strains according to the invention may particularly be of interest in breadmaking methods of the direct scheme type (NO-TIME DOUGH) and of the indirect scheme type (“SPONGE and DOUGH”) with non sweetened or slightly sweetened dough, with or without a mould inhibitor. Their use is however not limited to the specific applications mentioned above and hereafter. A “NO-TIME DOUGH” scheme or “direct scheme” does practically not include any first fermentation between intensive kneading and division of the dough, the obtained dough rolls being fermented in a mould between 35° C. and 40° C. and then baked.

A “SPONGE and DOUGH” scheme is a widely practiced breadmaking method with two fermentation steps:
a first step or “SPONGE” step, which corresponds to the fermentation of a dough comprising 50-70% of the total flour applied, a portion of the water and the totality of the yeast for several hours, generally about four hours,
a second step, or “DOUGH” step, in which the SPONGE after the fermentation described above is combined with the remainder of the flour, the remainder of the water and the other ingredients of the dough (including the totality of the saccharose), the mixture thereby formed is kneaded, divided, put into a mould and fermented, and then baked, this second fermentation in the mould corresponding to the “proof” and its duration being the proof time.

Advantageously, the yeasts from the yeast strains according to the invention give shorter proof time values than those obtained with the yeasts from the reference strain in a breadmaking test with a direct scheme (NO-TIME DOUGH). The percentages are expressed in so-called baker's percentages, the so-called baker's percentage being a calculation method applied to the ratios of the ingredients in which the total mass of the flour always represents 100% and the mass of the other ingredients of the dough is calculated relative to this flour mass. The invention is further directed to a baker's dough containing baker's yeast as defined above. In an advantageous embodiment, the baker's dough is non sweetened or slightly sweetened dough.

In another embodiment, the baker's dough contains mould inhibitors, preferably as weak organic acids (for example having a pKa from 3 to 6) and/or their salts, or further preferably as propionates. The mould or anti-fungal inhibitor may be selected from acetic acid, propionic acid, sorbic acid or their salts. In a preferred embodiment according to the invention, the mould inhibitor is calcium propionate. This inhibitor, in particular calcium propionate, is preferably incorporated in the dough at a concentration of 0.2-0.5% by mass based on the flour mass, in particular in an amount of 0.4% by mass, based on the flour mass.

The invention is further directed to a method of preparing a baker's dough comprising a step of fermentation by yeast, as defined above. The invention is further directed to a method of preparing a breadmaking baked product comprising a step of baking a baker's dough as defined above. The invention finally is directed to a breadmaking product which is obtainable by the method as described above.

The following examples illustrate the invention without limiting it. The examples provide the characterization of the strains according to the invention and of the yeasts obtained from these strains.

### EXAMPLE 1

### Measurement of Maltase Activity

Materials and Methods

The maltase activity is measured by assaying the release of p-nitrophenol following the action of maltase on p-nitrophenyl-α-D-glucopyrannoside, as described in Houghton-Larsen and Anders Brandt, Appl. Environ. Microbiol. 2006, pp 7176-7182.

Pre-Cultivation of the Yeasts

The yeasts are cultivated in a YPG medium containing 2% glucose overnight at 30° C. with stirring.

Cultivation of the Yeasts

The pre-culture obtained above is centrifuged and the cells are washed before being sown in an amount of 1 mg of dry matter per mL in YPG medium containing 2% glucose or in YPM medium containing 2% maltose. After 4 hours of incubation, with stirring, at 30° C., the cells are harvested and a cell suspension with 20 mg of dry yeast material per mL is prepared. 1 mL of said cell suspension is taken up for milling the cells. After milling the cells, the supernatant is then recovered for assaying maltase activity.

Measurement of Maltase Activity

The supernatant obtained above is diluted between 5 and 400 times for the assay. 1.4 mL of substrate at a concentration of 4 mM in a phosphate buffer at pH 6.8 is added to 100 μL of a supernatant dilution. After 10 minutes of incubation at 30° C., the reaction is stopped by adding 1 mL of a 10% Na₂CO₃ solution. The solution is centrifuged for 5 minutes at 4,000 rpm and the absorbance of the supernatant is measured at 400 nm.

The p-nitrophenol concentration in the supernatant is inferred from absorbance values obtained with one gram of p-nitrophenol between 100 and 800 nmol/mL. The results are then expressed in nanomoles of p-nitrophenol released per minute and per mg of proteins. The maltase activity of the reference NCYC995 strain, of the slow osmotolerant NCYC996 strain and of the slow CNCM I-2412 strain with strong induction in the presence of maltose is also measured.

Expression of the Results

The maltase activity of the tested strains is expressed at the same time:
as a percentage of the maltase activity of the reference NCYC995 strain:

$$\frac{( maltase activity of the tested strain )}{( maltase activity of the reference strain )} \times 100 , and$$

as a percentage of the difference of the maltase activity relatively to the reference strain NCYC995:

$$\frac{[ \begin{matrix}( maltase activity of the tested strain ) - \\ ( maltase activity of the reference strain )\end{matrix} ]}{( maltase activity of the reference strain )} \times 100$$

Results

The maltase activity measured under induction or repression conditions is illustrated in FIG. 1. Table 1 indicates the maltase activity under the induction condition (maltose).

TABLE 1
Maltase activity under induction condition

| | Maltase activity as a | % of the difference of the |
|---|---|---|
| | % of the activity of | maltase activity relatively |
| Strain | the NCYC995 strain | to the NCYC995 strain |
| I-3796 | 157 | +57 |
| I-3797 | 165 | +65 |
| I-3798 | 130 | +30 |
| NCYC996 | 63 | −37 |
| CNCM I-2412 | 143 | +43 |

As regards induction in the presence of maltose, the strains according to the invention show strong induction of the maltase activity in the presence of maltose: an increase of 30% for strain I-3798, 57% for strain I-3796 and 65% for strain I-3797 is observed relatively to the reference strain NCYC995. As a comparison, the slow strain NCYC996 has a 37% reduction of maltase activity relatively to the activity of the reference strain NCYC995. The slow strain CNCM I-2412 has a particular profile since it has a strong induction of the maltase activity in the presence of maltose.

Table 2 indicates the maltase activity under repression (glucose) condition.

TABLE 2
Maltase activity under repression condition

| | Maltase activity as a | % of the difference of the |
|---|---|---|
| | % of the activity of | maltase activity relatively |
| Strain | the NCYC995 strain | to the NCYC995 strain |
| I-3796 | 55 | −45 |
| I-3797 | 134 | +34 |
| I-3798 | 51 | −49 |
| NCYC996 | 26 | −74 |
| CNCM I-2412 | 10 | −90 |

The slow control strains NCYC996 and CNCM I-2412 exhibit strong repression of the maltase activity in the presence of glucose, with a maltase activity equal to 26% and 10% respectively of the maltase activity of the reference strain. As a comparison, strains I-3796 and I-3798 exhibit a maltase activity in the presence of glucose equal to 55% and 51% respectively of the maltase activity of the reference strain. Strains I-3796 and I-3798 are therefore rapid strains. The strain I-3797 has a maltase activity in the presence of glucose greater than that of the reference strain (equal to 134% of the maltase activity of the reference strain) and therefore has excellent non-repression of the maltase activity in the presence of glucose.

### EXAMPLE 2

### Yield and Fermentative Strength

Materials and Methods

Culture on a Molasses Plate

Pre-cultivation of the yeast strains to be tested is carried out by seeding 0.3 mg of the yeast strain on a Petri dish with a diameter of 90 mm containing 20 mL of YEG medium (2% glucose). The YEG medium contains 20 g/mL of glucose, 5 g/L of yeast extract and 30 g/L of agar-agar. After 16 hours of incubation at 30%, the yeast cells contained in the Petri dish are harvested.

The yeast cells harvested at the end of the pre-cultivation are sown in Petri dishes with a diameter of 140 mm, containing molasses, in an amount of 2 mg of dry yeast material per dish. The “molasses” medium contains 5 g/L of molasses, 0.5 g of (NH₄)₂HPO₄, 12.7 g/L of K₂SO₄, 5.8 g/L of Na₂SO₄, 30 g/L of agar-agar, at pH 5-5.5. After 20 h of incubation at 30° C., the yeast cells contained in the Petri dishes are harvested and washed. The yeast cells are re-suspended in 20 mL of de-mineralized water.

Semi-Continuous Cultivation (Fed-Batch)

The yeasts are cultivated in 7 litre fermenters, in a semi-continuous mode as described in the reference textbook “Yeast Technology”, 2^{nd} edition, 1991, G. Reed and T. W. Nagodawithana, published by Van Nostrand Reinhold, ISBN 0-442-31892-8. In this cultivation mode, the molasses are brought discontinuously into the fermenter.

Measurement of the Yield

The dry matter mass of harvested yeasts is determined.

The percentage yield is then calculated according to the following formula:

$$Yield = \frac{( produced yeast expressed with 32 \% of dry matter )}{( amount of molasses with 50 \% sugar )} \times 100$$

The yields are then expressed:
as a percentage of the yield obtained with the reference strain NCYC995:

$$\frac{( yield of the tested strain )}{( yield of the reference strain )} \times 100 , and$$

as a percentage of the yield difference relatively to the one obtained with the reference strain NCYC995:

$$\frac{[ ( Yield of the tested strain ) - ( yield of the reference strain ) ]}{( yield of the reference strain )} \times 100$$

Measurement of the Fermentative Strength

The fermentative strength is measured on dough rolls consisting of 20 g of flour and of a suspension of yeasts, in a fermentometer of the Burrows and Harrison type, over a period of 2 hours. For measuring the fermentative strength after cultivation on a molasses plate, the suspension of yeasts consists of 100 mg of dry yeast materials in 15 mL of water containing 27 g/L of NaCl and 4 g/L of SO₄(NH₄)₂. For measuring the fermentative strength after semi-continuous cultivation, the suspension of yeasts consists of 150 mg of dry yeast materials in 15 mL of water containing 27 g of NaCl and 4 g/L of SO₄(NH₄)₂.

In order to form the dough rolls, the mixture of flour and of yeast suspension is kneaded for 40 seconds in a kneading-trough, so as to obtain dough which is then placed in a water bath at 30° C. 13 minutes after kneading, the container containing the dough is sealed hermetically. The total amount of produced gas is measured in mL after 2 hours at 30° C.

The fermentative strength is measured under different dough conditions:
non sweetened dough, the composition of which is the one mentioned above (PN),
non sweetened dough in the presence of 0.4% of calcium propionate by mass on the basis of the flour mass (PN+),
sweetened dough containing 2 g of saccharose for 20 g of flour (PS), and
sweetened dough containing 2 g of saccharose for 20 of flour and 0.4% of calcium propionate by mass based on the flour mass (PS+).

The fermentative strength difference of the tested strains relatively to that of the reference strain NCYC995 is calculated as a percentage according to the following formula:

$$\frac{[ \begin{matrix}( fermentative strength of the tested strain ) - \\ ( fermentative strength of the reference strain )\end{matrix} ] \times 100}{( fermentative strength of the reference strain )}$$

Results

(i) Yield and Fermentative Strength After Cultivation on a Molasses Plate:

Table 3 indicates the obtained yields relatively to the reference strain NCYC995 after cultivation on a molasses plate.

TABLE 3
Yield after cultivation on a molasses plate

| | Yield in % of the yield | % of yield difference relatively |
|---|---|---|
| | obtained with the | to the one obtained with |
| Strain | NCYC995 strain | the NCYC995 strain |
| I-3796 | 101 | +1 |
| I-3797 | 97 | −3 |
| I-3798 | 93 | −7 |

The measurement of the yield after cultivation on a molasses plate is used in the pre-selection in order to eliminate the strains which have a yield reduction of greater than 10% relatively to the reference strain NCYC995. The results appearing in Table 3 actually show that the selected strains according to the invention, i.e. strains I-3796, I-3797 and I-3798 have a yield above 90% of the yield obtained with the reference strain NCYC995.

Table 4 indicates the fermentative strength difference on non sweetened dough (PN) of the tested strains relatively to that of the reference strain NCYC995, as a percentage, after cultivation on a molasses plate.

TABLE 4

| Strain | PN |
|---|---|
| I-3796 | +11 |
| I-3797 | +1 |
| I-3798 | +10 |

The pre-selection on a molasses plate aimed at eliminating the strains having a fermentative strength less than that of the reference strain NCYC995. The three strains according to the invention actually exhibit fermentative strengths greater than or equal to that of the reference strain.

(ii) Yield and Fermentative Strength After Semi-Continuous Cultivation:

In a second phase, the yield of the pre-selected strains is evaluated after semi-continuous cultivation, in order to select the strains based on yield values approaching those obtained under industrial production conditions. The results appearing in Table 5 show that the obtained yields with the strains according to the invention are very satisfactory, since they are greater than 90% of the yield obtained with the reference strain NCYC995. Now, said reference strain is known for allowing production of yeasts with excellent yields.

TABLE 5
Yield after semi-continuous cultivation

| | Yield as a % of the yield | % of yield difference relatively |
|---|---|---|
| | obtained with the | to the one obtained with |
| Strain | NCYC995 strain | the NCYC995 strain |
| I-3796 | 97 | −3 |
| I-3797 | 92 | −8 |
| I-3798 | 100 | 0 |

Concerning the fermentative strength, Table 6 indicates the fermentative strength difference of the tested strains relatively to that of the reference strain NCYC995, as a percentage, after semi-continuous cultivation.

TABLE 6

| Strain | PN | PN+ | PS | PS+ |
|---|---|---|---|---|
| I-3796 | +14 | nd | +26 | nd |
| I-3797 | +28 | +36 | +32 | +42 |
| I-3798 | +14 | +14 | +26 | +32 |

nd: not determined

The three tested strains show a fermentative strength increased by at least 14% relatively to the reference strain NCYC995. According to Table 6, the I-3797 strain has a very interesting fermentative strength both on a non sweetened and slightly sweetened dough (an increase by 28% and 32% respectively, relatively to the reference strain), which is further improved in the presence of calcium propionate (an increase by more than 35% in both types of dough). The I-3796 and I-3798 strains give a better fermentative strength on sweetened dough, with an increase of the fermentative strength by 26% for both strains, relatively to the reference strain. The fermentative strength of both of these strains on a non sweetened dough remains very interesting, with a 14% increase relatively to the reference strain.

### EXAMPLE 3

### Fermentative Activity in Breadmaking Tests

Materials and Methods

In a given breadmaking method, and with given recipes, the proof time difference is measured between a yeast obtained with the strain to be evaluated on the one hand, and a yeast obtained with the reference strain NCYC995 on the other hand, both yeasts being obtained with a same making method. The yeasts are cultivated in 20 liter fermenters, in a semi-continuous mode, as described in the reference textbook “Yeast Technology”, 2^{nd} edition, 1991, G. Reed and T. W. Nagodawithana, published by Van Nostrand Reinhold, ISBN 0-442-31892-8. By producing yeasts in a 20 liter fermenter, it is possible to obtain, after drying, a sufficient amount of dry yeasts for carrying out breadmaking tests. The fresh breadmaking yeasts with 32% of dry matter thus obtained are then dried by rapid careful drying in the presence of an emulsifier, 1.5% sorbitan monostearate.

The dry yeasts obtained are tested in a breadmaking method in a direct scheme (No Time Dough) in:
non sweetened dough at room temperature,
non sweetened dough at 18° C., and
slightly (10%) sweetened dough at room temperature.

The test procedure applied to the recipes above is the following:
weighing the 6 or 7 solid ingredients,
measuring the room temperature and the temperature of the flour,
adjusting the water temperature so as to obtain a dough temperature of 27° C.+/−0.5° C.,
placing the ingredients in a Mac Duffy® tank of a HobartA200® dough-trough,
slowly mixing at the first speed for 1 min,
starting kneading according to the following program:

1st speed for 5 min

let to rest for 5 min

2^{nd} speed for 5 min,
obtaining a dough having a temperature of 27° C.+/−0.5° C.,
checking the mass at 23° C. for 10 min,
dividing it into dough rolls of 320 g,
making a loose ball and covering,
letting it rest for 10 min,
shaping the dough,
putting the 320 g dough rolls into moulds (dimensions: base of the mould 185×75 mm; top of the mould 200×90 mm; mould height 75 mm),
determining the proof time or the proof time in a Stericult® incubator at 35° or 40° C. and with 90% relative humidity, the proof time being the time between when the dough is put into the incubator and the moment when the dough reaches a 85 mm height in the mould,
baking in a REED® tray oven at 190° C. for 22 min,
measuring the volume of the breads after cooling for at least one hour and estimating the score of the obtained breads.

Results

Table 7 indicates the difference between the proof time obtained with the dry yeasts from the strains according to the invention and that of dry yeasts from the reference strain NCYC995, this difference being expressed as a percentage. The proof times were measured after a breadmaking method in a direct scheme, on normal doughs or on slightly sweetened (containing 10% sugar) doughs, at room temperature. On non sweetened dough, the yeasts from the strains according to the invention have a proof time reduced by 17%-22% relatively to the yeasts from the reference strain: their fermentative activity is therefore greater than that of the yeasts for the reference strain. On slightly sweetened dough, the yeasts from the strains according to the invention have a proof time reduced by 14%-16% relatively to the yeasts from the reference strain: their fermentative activity is therefore greater than that of the yeasts from the reference strain.

TABLE 7

| | Direct scheme | Direct scheme with |
|---|---|---|
| Starting strain | without sugar | 10% sugar |
| I-3796 | −20% | −14 |
| I-3797 | −17% | −16 |
| I-3798 | −22% | −16 |

The dry yeasts respectively obtained for the strains CNCM I-3796, CNCM I-3797 and CNCM I-3798 give a shorter proof time than the one obtained with the yeasts from the strain NCYC995 and are therefore more rapid in attaining the desired bread volume.

1. A strain of Saccharomyces cerevisiae, selected from the strain deposited on Aug. 21, 2007 at the CNCM under number I-3796, the strain deposited on Aug. 21, 2007 at the CNCM under number I-3797 and the strain deposited on Aug. 21, 2007 at the CNCM under number I-3798.

2. A method of selecting improved breadmaking yeast strains, the method comprising:
(a) a step of sporulation and hybridization of yeast strains and/or of mutagenesis of yeast strains, in order to obtain hybrid and/or mutated strains,
(b) a first step of selecting hybrid and/or mutated strains which have:
a maltase activity in the presence of maltose greater than that of the NCYC995 strain, and
a maltase activity in the presence of glucose greater than or equal to 50% of the maltase activity of the NCYC995 strain,
in order to obtain strains with improved maltase activity,
(c) optionally, a step of pre-selecting strains among the strains having improved maltase activity, which give yeasts, after cultivation on a molasses plate, which have:
a yield greater than or equal to 90% of the yield obtained with the NCYC995 strain, and
a fermentative strength greater than or equal to that of the NCYC995 strain, and
(d) a second step of selecting strains among the strains having improved maltase activity and/or the strains obtained in the preceding step, which give yeasts, after semi-continuous cultivation, which have:
a yield greater than or equal to 90% of the yield obtained with the NCYC995 strain, and
a fermentative strength greater than that of the NCYC995 strain,
in order to obtain improved breadmaking yeast strains.

3. The method according to claim 2, wherein:
(a) the first selection step comprises the selection of hybrid and/or mutated strains which have:
a maltase activity in the presence of maltose increased by at least 20%, relatively to that of the NCYC995 strain, and/or
a maltase activity in the presence of glucose greater than or equal to 70%, of that of the NCYC995 strain, and/or
(b) the second selection step comprises selecting strains giving yeasts, after semi-continuous cultivation, which have:
a fermentative strength on a non sweetened and/or slightly sweetened dough increased by at least 10% relatively to that of the NCYC995 strain, and/or
a yield greater than or equal to 95% of the yield obtained with the NCYC995 strain.

4-12. (canceled)

13. The method according to claim 2, wherein:
(a) the first selection step comprises the selection of hybrid and/or mutated strains which have:
a maltase activity in the presence of maltose increased by at least 30% relatively to that of the NCYC995 strain, and/or
a maltase activity in the presence of glucose greater than or equal to 90% of that of the NCYC995 strain, and/or
(b) the second selection step comprises selecting strains giving yeasts, after semi-continuous cultivation, which have:
a fermentative strength on a non sweetened and/or slightly sweetened dough increased by at least 14% relatively to that of the NCYC995 strain, and/or
a yield greater than or equal to 98% of the yield obtained with the NCYC995 strain.

14. A strain of Saccharomyces cerevisiae which is obtainable by the method according to claim 2.

15. A strain of Saccharomyces cerevisiae derived from the strain according to claim 1, said derived strain further comprising at least one of the following:
a maltase activity in the presence of maltose increased by at least 20% relatively to that of the NCYC995 strain, and/or
a maltase activity in the presence of glucose greater than or equal to 50% of that of the NCYC995 strain,
during the production of yeasts, a yield greater than or equal to 90% of the yield obtained with the NCYC995 strain,
after cultivation on a molasses plate, a fermentative strength of the yeasts on a non sweetened dough greater than or equal to that of yeasts from the NCYC995 strain,
after semi-continuous cultivation, a fermentative strength of the yeasts on a non sweetened dough increased by at least 10% relatively to that of yeasts from the NCYC995 strain,
after semi-continuous cultivation, a fermentative strength of the strains on a slightly sweetened dough increased by at least 10% relatively to that of yeasts from the NCYC995 strain.

16. The strain of claim 15, further comprising at least one of the following:
a maltase activity in the presence of maltose increased by at least 30% relatively to that of the NCYC995 strain,
a maltase activity in the presence of glucose greater than or equal to 50% of that of the NCYC995 strain,
during the production of yeasts, a yield greater than or equal to 90% of the yield obtained with the NCYC995 strain,
after cultivation on a molasses plate, a fermentative strength of the yeasts on a non sweetened dough greater than or equal to that of yeasts from the NCYC995 strain,
after semi-continuous cultivation, a fermentative strength of the yeasts on a non sweetened dough increased by at least 14% relatively to that of yeasts from the NCYC995 strain,
after semi-continuous cultivation, a fermentative strength of the strains on a slightly sweetened dough increased by at least 14% relatively to that of yeasts from the NCYC995 strain.

17. A strain of Saccharomyces cerevisiae derived from the strain according to claim 14, said derived strain further comprising at least one of the following:
a maltase activity in the presence of maltose increased by at least 20% relatively to that of the NCYC995 strain,
a maltase activity in the presence of glucose greater than or equal to 50% of that of the NCYC995 strain,
during the production of yeasts, a yield greater than or equal to 90% of the yield obtained with the NCYC995 strain,
after cultivation on a molasses plate, a fermentative strength of the yeasts on a non sweetened dough greater than or equal to that of yeasts from the NCYC995 strain,
after semi-continuous cultivation, a fermentative strength of the yeasts on a non sweetened dough increased by at least 10% relatively to that of yeasts from the NCYC995 strain,
after semi-continuous cultivation, a fermentative strength of the strains on a slightly sweetened dough increased by at least 10% relatively to that of yeasts from the NCYC995 strain.

18. The strain of claim 17, further comprising at least one of the following:
a maltase activity in the presence of maltose increased by at least 30% relatively to that of the NCYC995 strain,
a maltase activity in the presence of glucose greater than or equal to 50% of that of the NCYC995 strain,
during the production of yeasts, a yield greater than or equal to 90% of the yield obtained with the NCYC995 strain,
after cultivation on a molasses plate, a fermentative strength of the yeasts on a non sweetened dough greater than or equal to that of yeasts from the NCYC995 strain,
after semi-continuous cultivation, a fermentative strength of the yeasts on a non sweetened dough increased by at least 14% relatively to that of yeasts from the NCYC995 strain,
after semi-continuous cultivation, a fermentative strength of the strains on a slightly sweetened dough increased by at least 14% relatively to that of yeasts from the NCYC995 strain.

19. Baker's yeast which is obtainable by cultivating the strain according to claim 1.

20. Baker's yeast which is obtainable by cultivating the strain according to claim 15.

21. Baker's yeast which is obtainable by cultivating the strain according to claim 17.

22. Baker's yeast according to claim 19, having:
a yield greater than or equal to 90% of the yield obtained with the NCYC995 strain, and/or
a breadmaking proof time shorter than the one obtained with the breadmaking yeasts from the NCYC995 strain, and/or
a resistance to drying greater than or equal to that of the NCYC995 strain.

23. Baker's yeast according to claim 22, including a yield greater than or equal to 95% of the yield obtained with the NCYC995 strain.

24. Baker's yeast according to claim 22, including a yield greater than or equal to 98% of the yield obtained with the NCYC995 strain.

25. Baker's yeast according to claim 19, wherein the yeast is a yeast selected from yeast creams, pressed yeasts and dry yeasts.

26. Baker's dough containing baker's yeast according to claim 19.

27. A method of preparing baker's dough comprising a fermentation step by yeast according to claim 19.

28. A method of preparing a baked breadmaking product comprising a step of baking baker's dough according to claim 26.

29. A breadmaking product which is obtainable by the method according to claim 28.
